(19) United States
(12) Patent Application Publication
(10) Pub. No.: US 2004/0067492 A1
(43) Pub. Date: Apr. 8, 2004

(54) Reverse transcription on microarrays

(76) Inventors: Allan Peng, Palo Alto, CA (US); James Robert Love JR., Millbrae, CA (US); Qianjin Hu, Castro Valley, CA (US)

Correspondence Address: Shantanu Basu, Morrison & Foerster LLP, 755 Page Mill Road, Palo Alto, CA, 94304-1018, US

(21) Appl. No.: 10/264,378

(22) Filed: Oct. 4, 2002

(51) Int. Cl. C12Q 1/68

(52) U.S. Cl. 435/6

(57) ABSTRACT

The present invention provides novel methods for detection of gene. A method for detecting RNA molecules of interest in which oligonucleotide primers uniquely complementary to specific RNA molecules are used as primers for reverse transcribing target RNAs with reverse transcriptase, or any enzyme that possesses reverse transcription activity, is provided. The invention eliminates the need for labeling or converting the target RNA by incorporating detectable labels in the elongation product of reverse transcription of the probe-bound target RNA in the sample.

### TECHNICAL FIELD OF THE INVENTION

The invention relates to the field of nucleic acid biology. Specifically, this invention relates to the field of detection of nucleic acids on microarrays. More specifically, this invention relates to the detection of specific RNA on microarrays using reverse transcription.

### BACKGROUND OF THE INVENTION

The pattern of gene expression in a particular living cell is characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis and detection of pathogens (such as viruses and microorganisms including bacteria, yeast and protozoa), (c) analysis of differential gene expression over time, between tissues and disease states, and (d) drug discovery and toxicology studies. For example, increase or decrease in the levels of expression of certain genes correlate with cancer biology as oncogenes are positive regulators of tumorigenesis, while tumor suppressor genes are negative regulators of tumorigenesis (Marshall, Cell, 64: 313-326 (1991); Weinberg, Science, 254: 1138-1146 (1991)). However, detection of small quantities of such expressed genetic materials represents a major challenge in biological research and clinical diagnosis.

A number of methods are known in the art for detecting and comparing gene expression levels in different biological sources. One standard method for such detection of RNA levels is the Northern blot. In this technique, RNA is extracted from the sample and loaded onto any of a variety of gels suitable for RNA analysis, which are then run to separate the RNA by size, according to standard methods (see, e.g., Sambrook, J., et al., Molecular Cloning, A Laboratory Manual. Cold Spring Harbor Laboratory Press, Cold Spring Harbor, N.Y. (2nd ed. 1989)). The gels are then blotted (as described in Sambrook, supra), and hybridized to probes for detection of RNAs of interest. Northern blots are not used very often for diagnostic purposes; they are used mainly in research.

Recent developments in DNA microarray technology make it possible to conduct a large scale assay of a plurality of target molecules on a single solid phase support. U.S. Pat. No. 5,837,832 (Chee et al.) and related patent applications describe immobilizing an array of oligonucleotide probes for hybridization and detection of specific nucleic acid sequences in a sample. Target polynucleotides of interest are hybridized to the DNA chip and the specific sequence variations detected based on the target polynucleotides' preference and degree of hybridization at discrete probe locations. One important use of arrays is in the analysis of differential gene expression, where the expression of genes in different cells, often a cell of interest and a control cell, is compared and any differences in gene expression among the respective cells are identified. Such information is useful for the identification of the types of genes expressed in a particular cell or tissue type in a known environment. The economics of arrays provides a high density design platform of microarrays for rapid detection of transcription events for large numbers of genes.

Typically, RNA from the sample of interest is subjected to reverse transcription to obtain labeled cDNA. See U.S. Pat. No. 6,410,229 (Lockhart et al.) The cDNA is then hybridized to oligonucleotides or cDNAs of known sequence arrayed on a chip or other-surface in a known order. The location of the oligonucleotide to which the labeled cDNA hybridizes provides sequence information on the cDNA, while the amount of labeled hybridized RNA or cDNA provides an estimate of the relative representation of the RNA or cDNA of interest. See Schena, et al. Science 270:467-470 (1995). For example, use of a cDNA microarray to analyze gene expression patterns in human cancer is described by DeRisi, et al. (Nature Genetics 14:457-460 (1996)).

U.S. Pat. No. 5,888,819 (Goelet et al.) discloses a method of determining the identity of a nucleotide base at a specific position in a nucleic acid of interest, involving the use of at least two different terminators of a nucleic acid template-dependent primer extension reaction to determine the identity of a nucleotide base at a specific position in a nucleic acid of interest. Since this method involves the use of labeled terminators in the absence of dATP, dCTP, dGTP, or dTTP, the primer can be extended by at most a single nucleotide.

U.S. Pat. No. 6,352,829 (Chenchik et al.) discloses a method of analyzing differences in the RNA profiles between two or more physiological sources, by contacting the RNA from each source with a representational number of gene-specific primers, and then hybridizing the resulting subpopulation of labeled cDNA to an array of oligonucleotide probes.

Several problems are associated with the current hybridization-based sequence variation assays, thus limiting their applications. See review by Hacia (1999) Nature Genetics Supp. 21:42-47. For example, accuracy of the hybridization assay remains poor. The same experimental approach applied to any two sequences can yield results with vastly different accuracy. Limitations of microarray analysis also include the difficulty of detecting nucleic acids that are available for microarray detection only in small volumes and small quantities. As any technology based on nucleic acid hybridization, the sensitivity of the microarray hybridization is limited in large by the number of target nucleic acids available, i.e., the abundance of the gene expression.

Presently, these limitations are overcome to a certain extent by amplifying and/or detecting a labeling signal (e.g., a fluorescent tag) that is attached to the nucleic acid target. A weakness of these methods is that to detect the target RNA it must first be labeled, usually through conversion to labeled cDNA. The RNA labeling methods are expensive, time-consuming and are likely to alter the detection results for relative abundance of specific mRNAs due to sequence-specific differences in efficiency of labeling. It would be a significant advantage in the field to develop more efficient and sensitive ways to detect one or more target RNAs on a micro array.

In some methods, reverse transcription is performed in the presence of natural nucleotides and the resulting RNA/DNA duplex detected directly by antibodies against a RNA-DNA duplex. U.S. Pat. No. 6,277,579 (Lazar et al.) describes a method of detecting target RNA molecules of interest (without labeling them) in which an array of reverse transcription primers, each primer unique to an RNA molecule of interest, is used for reverse transcribing the RNA with a reverse transcriptase. An anti-double stranded RNA:DNA hybrid antibody is used to detect the hybrid on the solid phase. The RNA:DNA hybrid antibody for capture or detection is prepared by the method of Kitawaga, Y. and Stollar, B. D., Mol. Immunology 19:413-420 (1982) or according to the method set forth in U.S. Pat. No. 4,732,847 (Stuart et al.). Since detection of the RNA/DNA hybrid is carried out by the use of an antibody specific for RNA/DNA hybrids, the reverse transcriptase for use in this method must be tailored to lack an RNase H function.

Preferably, the label of the anti-hybrid antibody is an enzyme, a fluorescent molecule or a biotin-avidin conjugate and is non-radioactive. The label can then be detected by conventional means well known in the art such as a calorimeter, a luminometer, or a fluorescence detector.

There exists a need for rapid high throughput analysis of hundreds to hundreds of thousands of RNA targets on microarrays with greater sensitivity and higher accuracy. There exists a need for improving the efficiency and accuracy of detection of gene expression levels by avoiding the drawbacks associated with labeling the target RNA, while facilitating a simple, rapid and accurate detection of RNA levels in a sample.

### SUMMARY OF THE INVENTION

The present invention provides novel methods for detection of gene expression with greater sensitivity, better accuracy and less time consumption, as compared to conventional hybridization-based approaches.

The current invention relates to a method of detecting RNA molecules of interest in which oligonucleotide primers uniquely complementary to specific RNA molecules of interest are used as primers for reverse transcribing such RNAs with reverse transcriptase, or any enzyme that possesses reverse transcriptase activity.

In one aspect, the invention eliminates the need for labeling or converting the target RNA by incorporating the detectable label in the elongation product of reverse transcription of the target RNA in the sample.

In one aspect, the invention enhances the accuracy of detection by using shorter primers for the reverse transcriptase reaction.

In another aspect, the invention eliminates the need for using specialized reverse transcriptase (RT) enzymes and can be used with reverse transcriptases that do or do not lack RNase H type nuclease activity.

In one aspect of the invention a method is provided for detecting a target RNA in a sample, the method comprising, (a) providing an array comprising a DNA primer comprising a sequence complementary to the target RNA; (b) contacting the primer with an RNA sample under conditions that promote specific hybridization between the primer and the target RNA; (c) incubating the primer-RNA heteroduplex with a reverse transcriptase enzyme, under conditions that allow reverse transcription of the RNA into cDNA such that a detectable label is incorporated into the cDNA; and (d) detecting the detectable label incorporated into the nascent cDNA strand synthesized by the reverse transcriptase, wherein the presence of the label in the cDNA is indicative of the presence of the target RNA in the sample. The method may further comprise: (e) comparing the presence or absence of a target RNA in a sample cell with respect to that in a reference cell. In another embodiment the method comprises: (e) determining a level of target RNA in a sample cell and comparing with the level in a reference cell.

In one embodiment, a plurality of DNA primers is provided for detecting a plurality of target RNAs in a sample, wherein each primer is complementary to a different target RNA molecule and further wherein each primer is located at a distinct and identifiable location on a solid support.

In one embodiment, the detectable label is selected from the group consisting of a radiolabeled molecule, a fluorescent molecule, and a chromogenic molecule. In another embodiment, detectable label is conjugated to a molecule that binds a second label incorporated into the nascent polynucleotide:

In one embodiment, the sample is derived from a cell and the presence or absence of at least one target RNA is indicative of a condition selected from the group consisting of an infection, a disease state, a predisposition to a disease state, a developmental, a physical, a chemical and a biological state.

In another embodiment, the primer is immobilized on a substrate by a covalent bond, such that the 3′ end of the primer is free. The covalent bond may be selected from the group consisting of a Schiff base, a photocleavable bond, an electrostatic bond, a disulfide bond, a peptide bond, a diester bond, and a selectively releasable bond. In one embodiment, the primer is immobilized on the substrate by a non-covalent coupling, such that the 3′ end of the primer is free. The non-covalent coupling may be selected from the group consisting of an electrostatic interaction, a hydrogen bonds, an antibody-antigen coupling, a biotin-avidin interaction, a biotin-streptavidin interaction, a Staphylococcus aureus protein A-IgG antibody F_{c} fragment interaction, and a streptavidin/protein A chimera.

In one embodiment, the substrate is selected from the group consisting of a plastic, a ceramic, a nylon, a polyester, a metal, a resin, a gel, a membrane, a nitrocellulose membrane, a plate, a bead, a thin film, a glass, a cylinder, a tagged bead, a magnetic bead, an optical fiber, and a woven fiber.

In several aspects of the invention, the RNA sample is isolated from a biological source, the RNA sample may be amplified following isolation from a biological source, the RNA sample is prepared by transcription in vitro, and the RNA sample comprises at least one expression control RNA.

In one embodiment, the primer comprises a sequence for a promoter of a DNA-dependent RNA polymerase selected from the group consisting of T3 RNA polymerase, T7 RNA polymerase, and SP6 RNA polymerase.

In one embodiment, the method further comprises: (e) amplifying the cDNA strand by a polymerase chain reaction (PCR) performed in situ using inter alia a Taq DNA polymerase, a Tth 1 DNA polymerase, a Vent DNA polymerase, a Pfu DNA polymerase, or a thermostable reverse transcriptase.

In one embodiment, a method is provided for detecting a target RNA in a sample, the method comprising, (a) providing a microarray comprising at least one DNA primer comprising a sequence complementary to a sequence of the target RNA; (b) contacting the microarray with an RNA sample under conditions that allow specific hybridization between the primer and the target RNA; (c) incubating the primer-RNA heteroduplex with a reverse transcriptase enzyme, under conditions that allow reverse transcription of the RNA into cDNA; and (d) detecting the cDNA-RNA heteroduplex synthesized by the reverse transcriptase enzyme, wherein presence of the cDNA-RNA heteroduplex is indicative of the presence of the target RNA in the sample, wherein the detection is not mediated by an antibody against a DNA-RNA hybrid. In some embodiments, the detectable label is conjugated to a molecule that binds the nascent polynucleotide. In one embodiment, this method further comprises: (e) providing a thermostable reverse transcriptase; and (f) performing a plurality of cycles of primer extension reactions under conditions wherein the temperature exceeds the melting temperature of a cDNA-RNA hybrid between each cycle.

In one embodiment, the cDNA-RNA heteroduplex is detected by a detectable reagent that specifically binds a double stranded polynucleotide, wherein the detectable reagent may be selected from the group consisting of an intercalating compound, a polynucleotide duplex-dependent fluorescence quenching compound, DAPI, ethidium bromide, thiazole orange, his-benzimide and acridine orange. In another embodiment, the detectable reagent is indirectly detectable by a second detectable molecule which binds the reagent.

The invention further provides a method for detecting a target RNA in a sample, the method comprising: (a) providing an array comprising a DNA primer comprising a sequence complementary to the target RNA; (b) contacting the primer with an RNA sample under conditions that promote specific hybridization between the primer and the target RNA; (c) incubating the primer-RNA heteroduplex with a thermostable reverse transcriptase enzyme, under conditions that allow reverse transcription of the RNA into cDNA such that a detectable label is incorporated into the cDNA; (d) performing at least two cycles of hybridization and reverse transcription, under conditions comprising a temperature higher than a melting temperature of the RNA:cDNA duplex; and (e) detecting the detectable label incorporated into the nascent cDNA strand synthesized by the reverse transcriptase, wherein the presence of the label in the cDNA is indicative of the presence of the target RNA in the sample.

In some embodiments of this method the hybridization and reverse transcription steps are performed in the presence of an RNase H inhibitor or the thermostable reverse transcriptase lacks an RNase H function.

A kit is provided for identifying sequence variations in a target polynucleotide as compared to a reference sequence, the kit comprising: a) an array of at least two oligonucleotide primers immobilized on a solid phase support, wherein each oligonucleotide primer is selected to comprise a sequence complementary to a target RNA, and occupies an identifiable and discrete area of the array; b) reagents suitable for a reverse transcription reaction on the array; and c) detection means for detecting a nascent cDNA strand on the array. The kit may further comprise a detectable label that is incorporated into the nascent cDNA strand during the reverse transcription reaction.

The methods of the invention allow detection of a relatively large signal based on specific hybridization of a relatively small primer. Relative to detection methods involving probe hybridization, this method allows increased specificity of detection. The methods also avoid prior labeling of the target RNA. Reverse transcription primers employed in the disclosed method are relatively shorter and therefore, less subject to non-specific hybridization. The specificity and signal generated by the disclosed method can be increased further by using a thermostable reverse transcriptase and performing multiple cycles of reverse transcription at a high temperature, such as a temperature around the melting temperature of the hybrid between the primer and target RNA.

### BRIEF DESCRIPTION OF THE DRAWING(S)

FIGS. 1A-1D depicts a schematic diagram of an embodiment of the method according to the invention.

FIGS. 2A-2B depict visualizations of gene expression profiles on microarrays using a labeled target RNA (FIG. 2A) and a labeled extension of a primer-probe where the label is introduced during reverse transcription.

### DETAILED DESCRIPTION OF THE INVENTION

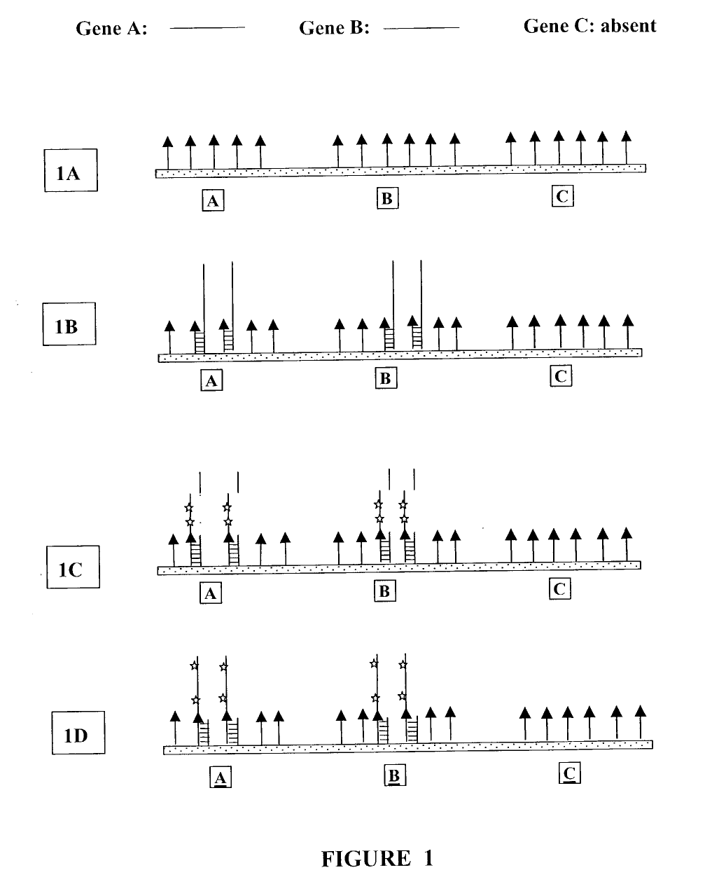

The invention relates to a method of detecting RNA molecules of interest in which oligonucleotide primers complementary to specific RNA molecules of interest are used as primers for reverse transcribing such RNAs with an enzyme having reverse transcriptase activity. The extended primers are labeled, directly or indirectly, and detected by appropriate means. A schematic diagram of an embodiment the process is illustrated in FIGS. 1A-1D. An array of primer/probes with free 3′ ends attached to discrete sites on a support is provided (FIG. 1A). The array is contacted with an RNA sample such that target RNAs that are complementary to the primer/probes are immobilized on the array by hybridization (FIG. 1B). The immobilized target RNAs are reverse transcribed in the presence of detectably labeled deoxyribonucleotides by an RNA-dependent DNA polymerase using the primer/probes as primers (FIG. 1C). Subsequent degradation of the target RNA by any RNase activity does not affect the detectable signal generated at each discrete site (FIG. 1D).

The sample containing the target RNA sequence(s) of interest can consist of a mixed population of nucleic acids, e.g., an mRNA extract from tissue, the end product of an in vitro transcription assay, predetermined amounts of a control RNA, etc.

In one embodiment, the oligonucleotide primers are immobilized to a solid phase support in microarray format, with each primer designed to hybridize to a nucleic sequence of interest, and occupying a discrete area of the array. The microarray on a solid phase support can comprise up to about 1,000,000 primers. As such, the method is useful for detecting up to about 1,000,000 different target RNA sequences. For most applications, a high number of groups are desirable, although it is clear that there is no lower limit to the number of groups that can be present on the solid support.

The immobilized cDNA that results from reverse transcription of target RNA can be directly detected in a variety of ways. First, reverse transcription can be performed in the presence of modified nucleotides capable of being incorporated into the synthesized cDNA during reverse transcription. For instance, the nucleotides can be detectably tagged by being radiolabeled or having incorporated fluorogenic or chromogenic dyes. Such nucleotides allow extension of a detectable nascent cDNA along the RNA template, in contrast to methods involving reverse transcription from arrayed primers in the presence of transcription terminators.

This method provides greater specificity of detection over current methods involving probe hybridization, since the reverse transcription can be accomplished with shorter primers and is less subject to nonspecific mispriming.

A. Definitions

As used herein a “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., phosphorothioates, phosphorodithioates, etc.), those containing pendant moieties, such as, for example proteins (including for e.g., nucleases, toxins, antibodies, signal peptides, poly-L-lysine, etc.),those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

The term “primer”, as used herein, refers to an oligonucleotide which is capable of acting as a point of initiation of polynucleotide synthesis along a complementary strand when placed under conditions in which synthesis of a primer extension product which is complementary to a polynucleotide is catalyzed. Such conditions include the presence of four different nucleotide triphosphates or nucleoside analogs and one or more agents for polymerization such as DNA polymerase and/or reverse transcriptase, in an appropriate buffer (“buffer” includes substituents which are cofactors, or which affect pH, ionic strength, etc.), and at a suitable temperature. A primer must be sufficiently long to prime the synthesis of extension products in the presence of an agent for polymerase. A typical primer contains at least about 5 nucleotides in length of a sequence substantially complementary to the target sequence, but somewhat longer primers are preferred. Usually primers contain about 15-26 nucleotides, but longer primers, up to 35 nucleotides, may also be employed.

A primer will always contain a sequence substantially complementary to the target sequence, i.e., the specific sequence to be detected, to which it can anneal. A primer may, optionally, also comprise a promoter sequence. The term “promoter sequence” defines a single strand of a nucleic acid sequence that is specifically recognized by an RNA polymerase that binds to a recognized sequence and initiates the process of transcription by which an RNA transcript is produced. In principle, any promoter sequence may be employed for which there is a known and available polymerase that is capable of recognizing the initiation sequence. Known and useful promoters are those that are specifically recognized by certain bacteriophage polymerases, such as bacteriophage T3, T7 or SP6. In embodiments of this invention, primers also serve the role of probes, typically immobilized on a substrate, to which a target RNA will hybridize.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to amplify or clone target polynucleotides. Since the position of each particular group of primers in the array is known, the identities of the target polynucleotides can be determined based on their binding to a particular position in the microarray.

A “linker” is a synthetic oligodeoxyribonucleotide which contains a restriction site. A linker may be blunt end-ligated onto the ends of DNA fragments to create restriction sites which can be used in the subsequent cloning of the fragment into a vector molecule.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical, chemical, or any other appropriate means. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

The term “support” refers to conventional supports such as beads, particles, dipsticks, fibers, filters, membranes and silane or silicate supports such as glass slides.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases.

As used herein, a “biological sample” refers to a sample of tissue or fluid isolated from an individual, including but not limited to, for example, blood, plasma, serum, spinal fluid, lymph fluid, the external sections of the skin, respiratory, intestinal, and genitourinary tracts, tears, saliva, milk, cells (including but not limited to blood cells), tumors, organs, and also samples of in vitro cell culture constituents.

The term “biological sources” as used herein refers to the sources from which the target polynucleotides are derived from. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

A. Target RNA

The RNA template used is generally purified from the biological source of interest. The initial mRNA sample may be derived from a physiological source including a single celled organism such as yeast, from a eukaryotic source, or a multicellular organism including plants and animals, particularly mammals and organs, tissues, and cells derived from the mammals such as from any bodily fluids (such as blood, urine, saliva, phlegm, gastric juices, etc.), cultured cells, biopsies, or other tissue preparations. Protocols for isolating nucleic acids from cells; tissues, organs and whole organisms are described in: Maniatis et al., Molecular Cloning: A Laboratory Manual (Cold Spring Harbor Press, 1989) which is incorporated herein by reference in its entirety. Such methods typically involve subjection of the original biological source to one or more of tissue/cell homogenization, nucleic acid/protein extraction, chromatography, centrifugation, affinity binding and the like. The method can also be performed on whole cell lysates without prior RNA isolation. Klebe, R. J. et al. (1996) BioTechniques 21, 1094. The method may also be performed on in vitro transcribed RNA, as well as RNA transcribed by T3, T7, SP6 or Qβ RNA-polymerases.

The disclosed method can be used to detect the presence of one or many specific RNA molecules which may be present in a sample such as RNA from different organisms (such as viruses, bacteria, fungi, plants, and animals), or RNA indicative of an infection, a disease state, or predisposition to a disease state. The method can also be used to detect a class of microorganisms or a group of related disease conditions.

The method can also be used with amplified target RNA. The polymerase chain reaction (PCR) procedure has been coupled to RNA transcription by incorporating a promoter sequence into one of the primers used for the PCR reaction and then, after amplification by the PCR procedure for several cycles, using the double-stranded DNA as template for the transcription of single-stranded RNA. (See Murakawa et al. DNA 7:827-295 (1988)). This combination of PCR and in vitro transcription (IVT) enables the generation of a relatively large amount of RNA from a small starting number of cells without loss of fidelity and is described in U.S. Pat. No. 6,271,002 (Linsley et al.) which is incorporated herein by reference in its entirety. Other methods of amplifying nucleic acid sequences are also commercially available. These methods include the ligation amplification reaction (LCR) described by Wu, D. Y and Wallace, R. B, Genomics 4:560-569 (1989) and Barringer, K. J., et al., Gene 89:117-122 (1990); transcription-based amplification reaction described by Kwoh, D. Y., et al., Proc. Natl. Acad. Sci. USA 86:1173-1177 (1989); Sequence Initiation Reaction (SIR), and continuous amplification reaction (CAR) RNA (see U.S. Pat. No. 5,981,179 by Lorinez et al.). In each case, the amplification product is RNA, or can be converted to RNA by incorporating a promoter for DNA-dependent RNA polymerase during the amplification process. This RNA is then detected by a method of the invention.

B. Array of Oligonucleotide Primers

1. Selecting Primers

The invention provides a prepared solid support comprising immobilized and separate groups of oligonucleotide primers. The primers can be selected or designed using for example a standard PCR primer selection program such as Primer3 from Massachusetts Institute of Technology (MIT). In an embodiment of the primers according to the present invention, they are used as “capture probes”, and are for this purpose immobilized on a substrate in order to capture the target nucleic acid contained in a biological sample. Primer/probes according to the invention must have a free 3′ end to enable extension of the reverse transcription product. In a preferred embodiment, primer/probes are immobilized to the substrate though the 5′ end.

The solid phase support can provide an area on which up to 1,000,000 or more primers may be immobilized in discrete areas according to a predetermined pattern. The prepared solid support can have an associated written or electronic record of the sequence of the primer or primer pairs at any given location on the support, and thus the location on the support of an immobilized target can be identified as well. Preferably, the amounts of primers (i.e. primer molecule numbers or primer concentration) will be about the same at each provided location on a given solid support (e.g. in a DNA microarray format having from 10, 100, 1000, to 10,000, to 100,000 up to about 1,000,000 primers to amplify or detect up to about 1,000,000 target polynucleotides).

The solid support can be prepared with primer sequences for a particular application based on the polynucleotides to be detected. The oligonucleotide primers can be of any length suitable for a particular reverse transcriptase, especially considering the sequence and quality of the target polynucleotides to be amplified. As an example, the primers can be from about 4 to about 100 nucleotides in length, in some embodiments 10, 20, or 30 nucleotides in length, and in other embodiments between 4 and 30 nucleotides in length. The primers are sufficiently specific to hybridize to complementary template sequence during the generation of labeled nucleic acids under conditions sufficient for first strand cDNA synthesis, under conditions known by those of skill in the art. The number of mismatches between the primer sequences and their complementary template (target RNA) sequences to which they hybridize during the generation of labeled nucleic acids generally do not exceed 15%, usually do not exceed 10% and preferably do not exceed 5%, as determined by FASTA (default settings). It is understood that a nucleic acid primer of the present invention may contain minor deletions, additions and/or substitutions of nucleic acid bases, to the extent that such alterations do not negatively affect the yield or product obtained to a significant degree.

Oligonucleotide primers can include the naturally-occurring heterocyclic bases normally found in nucleic acids (uracil, cytosine, thymine, adenine and guanine), as well as modified bases and base analogues. Any modified base or base analogue compatible with hybridization of the primer to a target sequence is useful in the practice of the invention.

The sugar or glycoside portion of the primer can comprise deoxyribose, ribose, and/or modified forms of these sugars, such as, for example, 2′-O-alkyl ribose. In a preferred embodiment, the sugar moiety is 2′-deoxyribose; however, any sugar moiety that is compatible with the ability of the primer to hybridize to a target sequence can be used.

In one embodiment, the nucleoside units of the primer are linked by a phosphodiester backbone, as is well known in the art. In additional embodiments, internucleotide linkages can include any linkage known to one of skill in the art that is compatible with specific hybridization of the primer including, but not limited to phosphorothioate, methylphosphonate, sulfamate (e.g., U.S. Pat. No. 5,470,967) and polyamide (i.e., peptide nucleic acids). Peptide nucleic acids are described in Nielsen et al. (1991) Science 254: 1497-1500, U.S. Pat. No. 5,714,331, and Nielsen (1999) Curr. Opin. Biotechnol. 10:71-75.

In certain embodiments, the primer can be a chimeric molecule; i.e., can comprise more than one type of base or sugar subunit, and/or the linkages can be of more than one type within the same primer. The primer can comprise a moiety to facilitate hybridization to its target sequence, as are known in the art, for example, intercalators and/or minor groove binders.

Variations of the bases, sugars, and internucleoside backbone, as well as the presence of any pendant group on the primer, will be compatible with the ability of the primer to bind, in a sequence-specific fashion, with its target sequence. A large number of structural modifications, both known and to be developed, are possible within these bounds. Moreover, synthetic methods for preparing the various heterocyclic bases, sugars, nucleosides and nucleotides which form the primer, and preparation of oligonucleotides of specific predetermined sequence, are well-developed and known in the art. A preferred method for oligonucleotide synthesis incorporates the teaching of U.S. Pat. No. 5,419,966.

The oligonucleotide primers can be designed with any special additional moieties or sequences that will aid and facilitate a particular manipulation, e.g. PCR, isolation of the amplified target polynucleotides, etc. For example, a primer can comprise sequences in addition to those that are complementary to the target sequence. Such sequences are normally upstream (i.e., to the 5′-side) of the target-complementary sequences in the primer. For example, sequences comprising one or more restriction enzyme recognition sites (so-called “linkers” or “adapters”), when present in a primer upstream of target-complementary sequences, facilitate cloning and subsequent manipulation of an amplification product. Other useful sequences for inclusion in a primer include those complementary to a sequencing primer and those specifying a promoter for a bacteriophage RNA polymerase, such as, for example, T3 RNA polymerase, T7 RNA polymerase and/or SP6 RNA polymerase. In some embodiments, the probe maybe partly single-stranded and partly double-stranded—wherein the single stranded region is complementary to the target RNA sequence and the double stranded region comprises an additional feature such as a restriction site, or a promoter for RNA polymerase.

Advantageously, the probes according to the present invention may have structural characteristics such that they allow the signal amplification, such structural characteristics being, for example, branched DNA probes as those described by Urdea et al. (Nucleic Acids Symp. Ser., 24:197-200 (1991)) or in the European Patent No. EP-0225,807.

In one aspect of the invention, the microarray primers are defined to cover an entire region of interest in the target polynucleotide. Preferably, the primer is designed to possess a sequence complementary to a region some distance downstream from the 5′ end of a target RNA. The distance is determined by the need to incorporate detectable labels or otherwise enable detection of the nascent DNA strand reverse transcribed off the hybridized primer. Multiple primers may be designed for a particular target RNA to account for polymorphism and/or secondary structure in the target RNA, redundancy of data and the like. When multiple target polynucleotides are to be detected according to the present invention, each primer or primer group corresponding to a particular target polynucleotide is situated in a discrete area of the microarray.

Probes may be in solution, such as in wells or on the surface of a micro-tray, or attached to a solid support. Examples of solid support materials which can be used include a plastic, a ceramic, a metal, a resin, a gel and a membrane. Useful types of solid supports include plates, beads, microbeads, hybridization chips, membranes, crystals, ceramics and self-assembling monolayers. A preferred embodiment comprises a two-dimensional or three-dimensional matrix, such as a gel or hybridization chip with multiple probe binding sites (Pevzner et al., J. Biomol. Struc. & Dyn. 9:399-410, 1991; Maskos and Southern, Nuc. Acids Res. 20:1679-84, 1992). Hybridization chips can be used to construct very large probe arrays which are subsequently hybridized with a target nucleic acid. Analysis of the hybridization pattern of the chip can assist in the identification of the target nucleotide sequence. Patterns can be manually or computer analyzed, but it is clear that positional sequencing by hybridization lends itself to computer analysis and automation. Algorithms and software, which have been developed for sequence reconstruction, are applicable to the methods described herein (R. Drmanac et al., J. Biomol. Struc. & Dyn. 5:1085-1102, 1991; P. A. Pevzner, J. Biomol. Struc. & Dyn. 7:63-73, 1989).

2. Coupling Probes to Solid Phase Support

Nucleic acid probes may be attached to the solid support by covalent binding such as by conjugation with a coupling agent or by, covalent or non-covalent binding such as electrostatic interactions, hydrogen bonds or antibody-antigen coupling, or by combinations thereof. Following attachment, the 3′ end of the probe must remain free for elongation during the reverse transcription reaction. Typical coupling agents include biotin/avidin, biotin/streptavidin, Staphylococcus aureus protein A/IgG antibody F_{c} fragment, and streptavidin/protein A chimeras (T. Sano and C. R. Cantor, Bio/Technology 9:1378-81 (1991)), or derivatives or combinations of these agents. Nucleic acids may be attached to the solid support by a photocleavable bond, an electrostatic bond, a disulfide bond, a peptide bond, a diester bond or a combination of these sorts of bonds. The array may also be attached to the solid support by a selectively releasable bond such as 4,4′-dimethoxytrityl or its derivative. Derivatives which have been found to be useful include 3 or 4[bis-(4-methoxyphenyl)]-methyl-benzoic acid, N-succinimidyl-3 or 4[bis-(4-methoxyphenyl)]-methyl-benzoic acid, N-succinimidyl-3 or 4[bis-(4-methoxyphenyl)]-hydroxymethyl-benzoic acid, N-succinimidyl-3 or 4[bis-(4-methoxyphenyl)]-chloromethyl-benzoic acid, and salts of these acids.

In a preferred embodiment, DNA probes are attached by the 5′ end by a Schiff Base reaction using a substrate containing primary aldehyde groups attached covalently to the glass surface (ArrayIt™ SuperAldehyde Substrate, TeleChem International Inc., Sunnyvale, Calif.). A 5′ amino-linker is covalently attached to oligonucleotide probes by modification of the oligonucleotides directly during oligonucleotide synthesis. Examples of 5′ amino-modifications suitable for the surface chemistry include a NH₂(CH₂)₆ linker, a photocleavable C6 amino-modifier, and a PC Amino-Modifier Phosphoramidite (Glen Research Corp., Sterling, Va.). The 5′ amino-linker allows selective binding of the amino-containing DNA to silylated slides through a Schiffs base reaction with aldehyde groups on the chip surface. Primary amino linkers on the DNA react with the aldehyde groups on the substrate forming covalent bonds. Attachment is stabilized by a dehydration reaction (drying in low humidity) which leads to Schiff base formation. Specific and covalent 5′-end attachment provide highly stable and accessible attachment of DNA for gene expression and genotyping applications. Attachment by the 5′ end leaves the 3′ end of the probe free for elongation by reverse transcriptase.

Binding may be reversible or permanent where strong associations would be critical. In addition, probes may be attached to solid supports via spacer moieties between the probes of the array and the solid support. Useful spacers include a coupling agent, as described above for binding to other or additional coupling partners, or to render the attachment to the solid support cleavable.

Cleavable attachments may be created by attaching cleavable chemical moieties between the probes and the solid support such as an oligopeptide, oligonucleotide, oligopolyamide, oligoacrylamide, oligoethylene glycerol, alkyl chains of between about 6 to 20 carbon atoms, and combinations thereof. These moieties may be cleaved with added chemical agents, electromagnetic radiation or enzymes. Examples of attachments cleavable by enzymes include peptide bonds which can be cleaved by proteases and phosphodiester bonds which can be cleaved by nucleases. Chemical agents such as β-mercaptoethanol, dithiothreitol (DTT) and other reducing agents cleave disulfide bonds. Other agents which may be useful include oxidizing agents, hydrating agents and other selectively active compounds. Electromagnetic radiations such as ultraviolet, infrared and visible light cleave photocleavable bonds. Attachments may also be reversible such as, for example, using heat or enzymatic treatment, or reversible chemical or magnetic attachments. Release and reattachment can be performed using, for example, magnetic or electrical fields.

The solid phase support of the present invention can be of any solid materials and structures suitable for supporting nucleotide hybridization and synthesis. Preferably, the solid phase support comprises at least one substantially rigid surface on which the primers can be immobilized and the reverse transcriptase reaction performed. The solid phase support can be made of, for example, glass, synthetic polymer, plastic, hard non-mesh nylon or ceramic. Other suitable solid support materials are known and readily available to those of skill in the art. The size of the solid support can be any of the standard microarray sizes, useful for DNA microarray technology, and the size may be tailored to fit the particular machine being used to conduct a reaction of the invention.

The substrates with which the polynucleotide microarray elements are stably associated may be fabricated from a variety of materials, including plastics, ceramics, metals, acrylamide, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Substrates may be two-dimensional or three-dimensional in form, such as gels, membranes, thin films, glasses, plates, cylinders, beads, magnetic beads, optical fibers, woven fibers, etc. A preferred form of array is a three dimensional array. A preferred three dimensional array is a collection of tagged beads. Each tagged bead has different primers attached to it. Tags are detectable by signaling means such as color (Luminex, Illumina) and electromagnetic field (Pharmaseq) and signals on tagged beads can be even be remotely detected (e.g., using optical fibers).

The arrays may be produced according to any convenient methodology, such as preforming the polynucleotide microarray elements and then stably associating them with the surface. A number of different array configurations and methods for their production are known to those of skill in the art and disclosed in U.S. Pat. No. 5,445,934 (in situ synthesis by photolithography), U.S. Pat. No. 5,532,128 (solid phase detection by electrical distribution differential); U.S. Pat. No. 5,384,261 (in situ synthesis by mechanically directed flow paths); and U.S. Pat. No. 5,700,637 (synthesis by spotting, printing or coupling); the disclosure of which are herein incorporated in their entirety by reference.

The solid support can be provided in or be part of a fluid containing vessel. For example, the solid support can be placed in a chamber with sides that create a seal along the edge of the solid support so as to contain the reverse transcriptase reaction on the support. In a specific example the chamber can have walls on each side of a rectangular support to ensure that the reverse transcription mixture remains on the support and also to make the entire surface useful for providing the primers.

The reverse transcription primer can be captured (Synen et al. Nucleic Acids Res. 14:5037 (1986)), adhered, or covalently coupled (e.g., by Schiff base formation) to a solid support (substrate) for the primer. The oligonucleotide primers of the invention are affixed, immobilized, provided, and/or applied to the surface of the solid support using any available means to fix, immobilize, provide and/or apply the oligonucleotides at a particular location on the solid support. For example, photolithography (Affymetrix, Santa Clara, Calif.) can be used to apply the oligonucleotide primers at particular position on a chip or solid support, as described in the U.S. Pat. No. 5,919,523, U.S. Pat. No. 5,837,832, U.S. Pat. No. 5,831,070 and U.S. Pat. No. 5,770,722, which are incorporated herein by reference. The oligonucleotide primers may also be applied to a solid support as described in Brown and Shalon, U.S. Pat. No. 5,807,522 (1998). Additionally, the primers may be applied to a solid support using a robotic system, such as one manufactured by Genetic MicroSystems (Woburn, Mass.), GeneMachines (San Carlos, Calif.) or Cartesian Technologies (Irvine, Calif.).

Alternatively, the polynucleotide microarray elements comprising the unique gene specific sequence and the standard sequences may be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array may be produced by hand or using available devices (slot blot or dot blot apparatus), materials (any suitable solid support), and machines (including robotic instruments) and may contain, for example, 8, 24, 96, 384, 1536 or 6144 oligonucleotides, or any other multiple between two and one million which lends itself to the efficient use of commercially available instrumentation.

Another method for coupling DNA to beads uses specific ligands attached to the end of the DNA to link to ligand-binding molecules attached to a bead. Possible ligand-binding partner pairs include biotin-avidin/streptavidin, or various antibody/antigen pairs such as digoxygenin-antidigoxygenin antibody (Smith et al., “Direct Mechanical Measurements of the Elasticity of Single DNA Molecules by Using Magnetic Beads,” Science 258:1122-1126 (1992)).

Covalent chemical attachment of the DNA to the support can be accomplished by using standard coupling agents to link the 5′-phosphate on the DNA to coated microspheres through a phosphoamidate bond. Methods for immobilization of oligonucleotides to solid-state substrates are well established. See Pease et al., Proc. Natl. Acad. Sci. USA 91(11):5022-5026 (1994). A preferred method of attaching oligonucleotides to solid-state substrates is described by Guo et al., Nucleic Acids Res. 22:5456-5465 (1994). Immobilization can be accomplished either by in situ DNA synthesis (Maskos and Southern, Nucleic Acids Research, 20:1679-1684 (1992) or by covalent attachment of chemically synthesized oligonucleotides (Guo et al., supra) in combination with robotics arraying technologies.

3) Controls for Expression Level.

Expression level controls are probes that hybridize specifically with constitutively expressed genes in the biological sample. Expression level controls are designed to control for the overall health and metabolic activity of a cell. Examination of the co-variation of an expression level control with the expression level of the target nucleic acid indicates whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse is also true. Thus where the expression levels of both an expression level control and the target gene appear to decrease or increase simultaneously, the change may be attributed to changes in the metabolic activity of the cell as a whole, and not to differential expression of the target gene. Conversely, where the expression levels of the target gene and the expression level control do not vary concurrently, the variation in the expression level of the target gene is attributed to differences in expression of that gene and not to overall variations in the metabolic activity of the cell.

Virtually any constitutively expressed gene provides a suitable target for expression level controls. Typically expression level control probes have sequences complementary to subsequences of constitutively expressed “housekeeping genes” including, but not limited to the beta-actin gene, the transferrin receptor gene, the GAPDH gene, and the like.

For the purpose of the invention, target polynucleotides are RNA. Examples of target polynucleotide include, but are not limited to mRNA, hnRNA and viral RNA. mRNA target polynucleotides can be directly used as templates for amplification mediated by reverse transcriptase. Following the completion of chain elongation originated from each immobilized primer site, the hybridized RNA template strand can be destroyed by, for example, RNase H, leaving the nascent complementary DNA strand affixed to the solid phase support. If a second primer (either specific or universal) is present in the solution phase, the first nascent cDNA strand will serve as a template for synthesizing another nascent strand, thereby forming a double-stranded nascent DNA molecule at each immobilized primer site or binding two immobilized primers.

Multiple target polynucleotides of the invention can be from one single biological source or, alternatively, from multiple biological sources such as different species or tissues. For example, a population of target polynucleotides isolated from a healthy individual can be mixed in one reverse transcriptase reaction with another population of target polynucleotides isolated from a patient with a disease of interest, under conditions that would allow distinguishing amplified products of the two sources by detection methods known in the art, as described in detail above. Therefore, the present invention can be used for a cross-species comparative analysis of target polynucleotides.

Detectable Labels

The detection of the target RNA is carried out by detecting the polynucleotides or oligonucleotides generated by reverse transcription of a target RNA captured on a site in the array by hybridization to a primer/probe. The reverse transcribed cDNA first strand is formed by extension of the primer/probe and nucleotides in the extended cDNA strand may be labeled with radioisotopes, chemiluminescent compounds, heavy metal atoms, spectroscopic markers, magnetic markers, linked enzymes, fluorescent labels, and the like. In one embodiment, the dNTPs are labeled such that the synthesized cDNA strand is labeled. A labeled entity comprises a member of a signal producing system and is thus detectable, either directly or in combination with one or more additional members of a signal producing system. Examples of directly detectable labels include isotopic and fluorescent moieties incorporated into a nucleotide monomer, usually by covalent linkage. Isotopic labels of interest include ³²P, ³³P, ³⁵S, ³H, ¹⁴C, ¹²⁵I, etc. and non-isotopic labels include biotin, acetylaminofluorene, digoxigenin, 5-bromodesoxyuridine, fluorescein, etc. Labels can be introduced with a directly or indirectly detectable marker according to procedures which are well known in the art, including non-isotopic methods such as via direct or indirect attachment of mass spectroscopy labels, fluorochromes or enzymes, or by various chemical modifications of the nucleic acid fragments that render them detectable immunochemically or by other affinity reactions.

Fluorescent moieties or labels of interest include coumarin and its derivatives, e.g. 7-amino-4-methylcoumarin, aminocoumarin; bodipy dyes, such as Bodipy Fla., cascade blue; fluorescein and its derivatives, e.g. fluorescein isothiocyanate, Oregon green; rhodamine dyes, e.g. Texas red, tetramethylrhodamine, eosins and erythrosins; cyanine dyes, e.g. Cy3 and Cy5; macrocyclic chelates of lanthanide ions, e.g. quantum dye™; and fluorescent energy transfer dyes, such as thiazole orange-ethidium heterodimer, TOTAB, etc. A fluorescent label may be introduced directly as dye-bearing nucleotides, or bound after amplification using dye-streptavidin complexes to incorporated biotin containing nucleotides. For fluorometric labels, the method typically uses light input near the excitation maximum and collects near the emission maximum (plus or minus about 10 nanometers is acceptable in most cases). With lasers, excitation can occur far from the excitation maximum. In some embodiments, the fluorescent labels are chosen such that they absorb light at wavelengths greater than 250 nm, preferably at wavelengths greater than about 350 nm, and fluoresce at wavelengths about 10 nm higher than the absorption wavelength. The fluorescent label may, for example, be fluorescein (absorption maximum of 488 nm), dichloro-fluorescein (absorption maximum of 525 nm), hexachloro-fluorescein (absorption maximum of 529 nm), BODIPY™ (absorption maximum of 530 nm), ROX (absorption maximum of 550 nm), tetramethylrhodamine (absorption maximum of 550 nm), rhodamine X (absorption maximum of 575 nm), Cy2™ (absorption maximum of 505 nm), Cy3™ (absorption maximum of 550 nm), Cy5™ (absorption maximum of 650 nm), Cy7™ (absorption maximum of 750 nm), IRD40 (absorption maximum of 785 nm), and the like. See Smith et al. (1986) Nature 321: 647-649. Fluorescent conjugates of nucleosides or nucleotides useful for labeling reverse transcription products are described in U.S. Pat. No. 6,340,767 (Bazin et al.) which is incorporated herein by reference.

Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugated antibody. The label is subsequently detected by colorimetry or chemiluminescence as described by Coutlee, et al., J. Clin. Microbiol. 27:1002-1007 (1989). In one embodiment, bound alkaline phosphatase is detected by chemiluminescence with a reagent such as a Lumi-Phos™ S30 reagent (Lumigen, Detroit, Mich.) using a detector such as an E/Lumina™ luminometer (Source Scientific Systems, Inc., Garden Grove, Calif.). For each sample of RNA, one generates labeled cDNAs with identical labels.

Particularly useful labels are enzymes, enzyme substrates, fluorophore, radioisotopic compounds, chromophores, magnetically responsive compounds, antibody epitope-containing compounds, haptens, and the like. For instance, a label can be calorimetric, radioactive, chemiluminescent, enzymatic or fluorescent. For instance, SYBR Green II (Molecular Probes Co., Ltd.) may be used for the detection of single stranded DNA following digestion of the RNA template with RNase H. Other commonly used dyes include ethidium bromide, thiazole orange, bis-benzimide (Hoechst 33258, Hoechst AG) and acridine orange.

The label can also be indirectly attached via a linker. The linkers that are useful in the practice of the present invention are specifically designed to promote efficient binding of the binding ligand to the nucleic acids and functioning of the label attached thereto. The use of linkers is described generally in U.S. Pat. Nos. 4,582,789 and 5,026,840.

Detection of Target RNA

The targets and probes are used in a method for immobilizing a polynucleotide probe comprising the steps of: combining the probe stably associated with a surface of a solid support with a polynucleotide target, wherein the probe and target comprise complementary strands, under conditions wherein the probe and target hybridize and the target RNA is thereby immobilized; elongating the hybridized probe with reverse transcriptase and detecting the product of reverse transcription.

In a particular embodiment, the method further comprises the step of amplifying the immobilized probe using a thermostable reverse transcriptase. In another particular embodiment, the method further comprises the step of amplifying the immobilized probe and then detecting resultant amplified probe.

Suitable hybridization conditions are well known to those of skill in the art and reviewed in WO 95/21944 to Maniatis et al. (supra). Incubation conditions are adjusted so that hybridization occurs with precise complementary matches or with various degrees of less complementarity. The various reagents and buffered media necessary for first strand cDNA synthesis through reverse transcription of the primed RNAs can be purchased commercially from various sources including but not limited to Sigma, Life Technologies, Amersham, Boehringer-Mannheim. Buffered media suitable for first strand synthesis comprise buffering agents, such as Tris-HCl, HEPES-K, in concentrations typically ranging from 10 to 100 μM and supporting a pH range of 6 to 9; 0-200 mM concentrations of monovalent salts, such as KCl, NaCl, etc.; 1 to 10 mM concentrations of divalent salts such as MgCl₂, Mg(OAc)₂, MnCl₂, ZnCl₂, etc.; and additional components such as dithiothreitol (DTT), detergents, albumin, glycerol and the like. The conditions of the reagent mixture will be selected to promote efficient first strand synthesis. Typically the set of primers is first combined with the RNA sample at an elevated temperature ranging from 50° to 95° C., followed by a reduction in temperature to between 0 to 60° C. This ensures specific annealing of the primers to their corresponding RNAs in the sample. Following the annealing step, primed RNAs are combined with dNTPs and reverse transcriptase under conditions sufficient to promote reverse transcription and first strand cDNA synthesis of the primed RNAs. Detectable labels are incorporated into the first strand cDNA. In some embodiments, a scanner is used for detection or visualization to determine the levels and patterns of fluorescence or any other detectable label used. A detection system may be used to measure the absence, presence, and amount of hybridization for all of the distinct sequences simultaneously.

In detecting or visualizing the hybridization pattern, the intensity or signal value of the label on the nucleic acids probe, and the intensity or signal value of the different label from the standard sequence probes is obtained. The intensity or signal value corresponding to the standard sequence represents the total amount or unit value of hybridization in the experiment, and serves as a control. The signal for each element of the microarray is measured and compared to the unit value. The signal from the standard targets becomes an element-specific control against which the signal from the probe can be calibrated or numerically adjusted. The invention thus provides quantitative information on each element of the microarray. Another advantage of the invention is that the hybridization of the target sequences to one or more probe sequences and the standard sequences is not competitive thereby reducing noise in the results,

The hybridization pattern can be used to determine quantitative information about the genetic profile of the nucleic acids in the sample that was contacted with the array to generate the hybridization pattern, as well as the physiological source from which the labeled sample nucleic acid was derived. The data provides information about the physiological source from which the sample nucleic acid were derived, such as the types of genes expressed in the tissue or cell which is the physiological source, as well as the levels of expression of each gene, particularly in quantitative terms.

The present method can be used in comparing nucleic acid samples from two or more physiological sources to identify and quantify differences between the patterns thereby providing data on the differential expression of a particular gene in the physiological sources being compared. Thus the methods of the invention find use in differential gene expression assays for the analysis of a diseased and normal tissue, analysis of a different tissue or subtissue types, and the like. Thus, this data may be used for large scale correlation studies on the sequences, mutations, variants, or polymorphisms among samples.

In an alternative embodiment, the standard polynucleotide sequences are doped into the sample containing the distinct polynucleotides with controlled stoichiometry. The standard sequences and the distinct polynucleotides are thus not covalently attached. The standard sequences, preferably having sequences different than the sequences of the microarray elements, are present in known amounts and can thus be used as a calibrating agent in subsequent analysis. In the method, the sample containing the polynucleotides for use in the fabrication of the microarray is spiked with the standard sequences. The standard sequences serve as a normalization control. The sample, containing the polynucleotides from the gene and the standard sequences, is then deposited on the microarray, hybridized with the labeled sample and differently labeled standard probes, and the hybridization pattern analyzed as described above.

After the reverse transcription is completed in the presence of appropriate labeling reagents, the labeled target polynucleotides can be detected at each of the original primer locations on the microarray. Detecting the labeled target polynucleotides can be conducted by standard methods used to detect the labeled sequences, including for example, detecting labels that have been incorporated into the amplified or newly synthesized DNA strands. Thus, for example fluorescent labels or radiolabels can be detected directly. Other labeling techniques may require that a label such as biotin or digoxigenin that is incorporated into the DNA during strand synthesis be detected by an antibody or other binding molecule (e.g. avidin, streptavidin) that is either labeled or which can bind a labeled molecule itself. For example, a labeled molecule can be an anti-streptavidin antibody or anti-digoxigenin antibody conjugated to either a fluorescent molecule (e.g., fluorescein isothiocyanate, Texas red and rhodamine), or conjugated to an enzymatically activatable molecule. Whatever the label on the newly synthesized molecules, and whether the label is directly in the DNA or conjugated to a molecule that binds the DNA (or binds a molecule that binds the DNA), the labels (e.g. fluorescent, enzymatic, chemiluminescent, or calorimetric) can be detected by a laser scanner or a CCD camera, or X-ray film, depending on the label, or any other appropriate means for detecting the particular label.

The target RNA can be detected by using labeled nucleotides (e.g. dNTP-fluorescent label for direct labeling; dNTP-biotin or dNTP-digoxigenin for indirect labeling) which are incorporated into reverse transcribed DNA during the process. For indirectly labeled DNA, the detection is carried out by fluorescence or other enzyme conjugated streptavidin or anti-digoxigenin antibodies. The method employs detection of the targets by detecting incorporated label in the newly reverse transcribed complements of the RNA targets. For this purpose, any label that can be incorporated into DNA as it is polymerized can be used, e.g., fluoro-dNTP, biotin-dNTP, or digoxigenin-dNTP, as described above and known in the art. Reverse transcription conducted using one or more primers in solution provides the option to detect the amplified targets at locations on the solid support by detecting the elongated primers. Thus, where more than one primer is used, target strands from different sources can be differentially detected on a solid support.

In a differential expression system, reverse transcription products derived from different biological sources can be detected by differentially labeling the amplified strands based on their origins. In one aspect, a label is incorporated into the nascent strand during reverse transcription so that the overall sensitivity for comparison of differential expression is enhanced.

Detection of RNA-DNA Duplex

The product generated by reverse transcription of a target RNA immobilized on a microarray by hybridization to a primer is a DNA-RNA hybrid duplex. Preferably, use of an RNase H-negative reverse transcriptase stabilizes the resulting DNA-RNA hybrid. Fluorescence techniques are also known for the detection of nucleic acid hybrids. U.S. Pat. No. 5,691,146 describes the use of fluorescent hybridization probes that are fluorescence-quenched unless they are hybridized to the target nucleic acid sequence. U.S. Pat. No. 5,723,591 describes fluorescent hybridization probes that are fluorescence-quenched until hybridized to the target nucleic acid sequence, or until the probe is digested

The following references describe DNA intercalating fluorescent dimers and their physical characteristics: Gaugain et al., Biochemistry 17, 5071-5078, 1978; Gaugain et al., Biochemistry 17, 5078-5088, 1978; Markovits et al., Anal. Biochemistry 94, 259-269, 1979; Markovits, Biochemistry 22, 3231-3237, 1983; and Markovits et al., Nucleic Acids Res. 13, 3773-3788, 1985. Interaction of various intercalating compounds with nucleic acids is reviewed by Berman and Young, Ann. Rev. Biophys. Bioeng. (1986) 10:87-224. Retention of ethidium bromide on electrophoresis of the dye with DNA or RNA is described by Angemuller and Sayavedra-Soto, Biotechniques 8, 36, 1990 and Rosen and villa-Komaroff, Focus 12, 23, 1990.

Polycyclic compounds which find use include phenanthridines, acridines, porphyrins, phenylindoles, and bisbenzimides. Derivatives of these compounds which find use include, bis-(3,8-diamino-6-hydroxy-6-phenyl-5,6-dihydrophenanthridine, di-(7-hydropyridocarbazoles), tetraacridinylamine, hexa-acridinylamine, thiazole orange dimer, 5-(11-(2-methoxy-6-chloro-9-aminoacridinyl)-(4,8-diazaundecyl)-3,8-diamino -6-phenylphenan-thridinium chloride, and the like.

Compounds can be prepared from alkylene polyamines, where the alkylene groups are of from 2-10, usually 2-6 carbon atoms, and haloalkyl- or pseudohaloalkyl substituted fluorescent polycyclic aromatic compounds, e.g., phenanthridines or acridines, which may be substituted or unsubstituted, to provide for ternary or quaternary amino groups. The amino groups may be quarternized with any convenient alkylation agent, either before or after reaction with the fluorescent compound or may be prepared initially as ternary amines using alkylamines, where the alkyl group will be of from about 1-6, usually 1-3 carbon atoms. Illustrative of a compound would be N, N, N′, N″, N″,-pentamethyl-N, N′, N″-tris-(3,8-diamino-6-hydroxy-6-phenyl-5,6-dihydrophenanthridine).

The heteroduplex should provide for means for binding to another molecule. This can be achieved in a wide variety of ways, depending upon the manner in which the label is to be employed. For example, the heteroduplex may include biotin conjugated nucleotides, one or more biotins, where the biotin will bind to avidin or streptavidin (hereafter both will be referred to as “avidin”). The biotins may vary from one biotin per nucleotide to 0.1% of the nucleotides depending on the nature of the procedures, conditions, etc. Alternatively, any molecule may be employed, particularly a small organic molecule (less than about 2 kD) which is unlikely to be encountered in the sample of interest, where the small organic molecule has a specific receptor or antibody, particularly monoclonal antibody, to which it specifically binds. Thus, thyroxine, corticosteroids, estrogens, retinoic acid, mannose and the like may be used with proteins which bind specifically to such molecules. Alternatively, synthetic molecules may be employed for which antibodies have been produced, such as 2,4-dinitrophenyl, barbiturate, phosphatidylcholine, etc. These molecules may be included during synthesis of the DNA by being linked to an internal or terminal nucleotide, where the DNA is synthesized in accordance with conventional automatic procedures, or may be added after synthesis of the DNA by linking to either available hydroxyl or amino groups.

For additionally measuring the concentration of double stranded nucleic acid in accordance with the invention, the agent may be 4′,6-diamidino-2-phenylindole (DAPI), ethidium bromide (EtBr), thiazole orange, bisbenzimide (Hoechst 33258, product of Hoechst AG) and acridine orange. In addition, SYBR Green I (Molecular Probes Co., Ltd.) may be included.

Amplification by PCR in situ

In one embodiment of this invention, the cDNA strand generated by reverse transcription is further amplified by PCR in situ. For PCR, a reaction mixture comprising the appropriate target polynucleotides mixed with the reagents necessary for conducting the polymerase chain reaction (PCR) are placed in contact with each immobilized cDNA:RNA hybrid on the solid support. The appropriate target polynucleotides can be single stranded cDNA generated by reverse transcription of RNA templates, or an mRNA sample in which case the PCR amplification is coupled to the reverse transcription detection reaction. The reaction mixture contains an enzyme for facilitating the synthesis of a polynucleotide strand complementary to a target strand, e.g. a polymerase. Suitable polymerases include thermostable polymerase enzymes, such as a Taq DNA polymerase, Tth1 DNA polymerase, Vent DNA polymerase, Pfu DNA polymerase, and thermostable reverse transcriptase enzymes. The reaction mixture can also contain a label molecule capable of being incorporated into the nascent strands during polymerase chain reaction so that the amplified products can be detected on the solid support after the PCR. The label can be detected directly or indirectly according to methods well known in the art.

After the reagents for conducting the PCR contact the immobilized primers on the microarray, the microarray is placed in conditions that facilitate the PCR to take place, using for example an automated system such as an in situ PCR machine. The reaction conditions for the PCR procedure can be as recommended by the in situ PCR machine manual, and may be varied as appropriate given the nature of the templates being used or any other difficulties anticipated with the primers and template hybridization. Temperatures and number of cycles can be selected as recommended and as appropriate given the primer selection and the template sequences, and any other relevant factors. The in situ-type PCR reactions on the microarrays can be conducted essentially as described in e.g. Embretson et al, Nature 362:359-362 (1993); Gosden et al, BioTechniques 15(1):78-80 (1993); Heniford et al Nuc. Acid Res. 21(14):3159-3166 (1993); Long et al, Histochemistry 99:151-162 (1993); Nuovo et al, PCR Methods and Applications 2(4):305-312 (1993); Patterson et al Science 260:976-979 (1993).

Detection Kits

Also provided are kits for carrying out the invention, where such kits include one or more microarrays fabricated such that the primer-probe elements contain the distinct polynucleotide sequences and the standard polynucleotide sequence, labeled standard complements to the standard sequences and instructional material for carrying out the subject methodology. The kit may also include one or more additional components necessary for carrying out the gene expression of the subject invention, where such additional components include enzymes, e.g. polymerases, reverse transcriptases, endonucleases, exonucleases, dNTPs, buffers, and the like. The kit can include, e.g. the materials and reagents for detecting a plurality of target polynucleotides that are otherwise difficult to detect on a solid support. The kit can include e.g. a solid support, oligonucleotide primers for a specific set of target polynucleotides, polymerase chain reaction reagents and components, e.g. enzymes for DNA synthesis, labeling materials, and other buffers and reagents for washing. The kit may also include instructions for use of the kit to amplify specific targets on a solid support. Where the kit contains a prepared solid support having a set of primers already fixed on the solid support, such solid supports can be custom-made for individual kits depending on the target polynucleotides the customer desires to detect. The solid support provides the affixed primers in designated locations on the solid support.

### EXAMPLE 1

Isolation of Target RNA from Cells

Cells often are harvested in late log phase of growth. RNA may be isolated by the guanidinium thiocyanate method (Chomczynski and Sacchi, Anal Biochem. 162(1): 156-159 (1987)). After RNA isolation, the nucleic acids are precipitated with ethanol. The precipitates are pelleted by centrifugation and redissolved in water. The redissolved nucleic acids are then digested with RNase-free DNase I (Boehringer Mannheim, Inc.) following the manufacturer's instructions, followed by organic extraction with phenol:chloroform:isoamyl alcohol (25:24:1) and reprecipitation with ethanol.

The DNase I treated RNA is then pelleted by centrifugation and redissolved in water. The purity and concentration of the RNA in solution is estimated by determining optical density at wave lengths of 260 nm and 280 nm (Sambrook et al., Molecular Cloning: A Laboratory Manual, 2nd Edition (Cold Spring Harbor Laboratory Press, Cold Spring Harbor, N.Y., 1989)). A small aliquot of the RNA is separated by gel electrophoresis in a 3% formaldehyde gel with MOPS buffer (Sambrook et al., supra) to confirm the estimation of concentration and to determine if the ribosomal RNAs were intact. This RNA is referred to as total cell RNA.

### EXAMPLE 2

Attachment of Probes to Substrates by Schiff Base Formation

A protocol for printing and attaching single- and double-stranded nucleic acids to the SuperAldehyde (ArrayIt™) surface uses DNA modified with primary amines that form Schiffs base covalent bonds with reactive aldehydes on the Substrate surface: (1) first, a C6 amino modification (Glen Research) is attached to the 5′ end of each oligonucleotide; (2) the amino-linked DNAs are resuspended in 1× Micro-Spotting Solution Plus (TeleChem); (3) the amino-linked DNAs are printed onto SuperAldehyde Substrates (ArrayIt™) with Stealth Micro-Spotting Pins (TeleChem); (4) the Substrates are allowed to dry for 12 hrs at room temperature (˜25° C.) at 30% relative humidity; (5) the Substrates are rinsed twice in 0.2% SDS for 2 min at room temperature with vigorous agitation to remove unbound DNA; (6) the Substrates are rinsed twice in deionized H₂O for 2 min at room temperature with vigorous agitation; (7) the Substrates are transferred into deionized H₂O at 95-100° C. for 2 min to denature the DNA; (8) the Substrates are allowed to cool at room temperature for ˜5 min; (9) the Substrates are treated with a sodium borohydride solution for 5 min at room temperature to reduce free aldehydes (to prepare the sodium borohydride solution, 1.5 g NaBH₄ (Sigma) is dissolved in 450 ml phosphate buffered saline (PBS), then 133 ml of 100% ethanol is added to reduce bubbling and prepared immediately prior to use); (10) the Substrates are rinsed three times in 0.2% SDS for 1 min each at room temperature; (11) the Substrates are rinsed once in deionized H₂O for 1 min at room temperature; (12) the printed Substrates are dried by centrifugation for 1 min at 500×g. Processed Substrates are stable for one year at room temperature if stored under appropriate conditions. The printed microarray is then reacted with a target RNA sample. Microarray reactions are best performed under glass cover slips at a volume of 2.0 μl per cm² of cover slip. For nucleic acid hybridization reactions, buffers typically contain 5×SSC or 6×SSPE and 0.1 % SDS and are performed at 37°-65° C. depending on the nature of the targets and probes.

### EXAMPLE 3

Reverse Transcription on Microarrays

RNA-dependent DNA polymerases (reverse transcriptases) are used for the first strand cDNA synthesis step. Examples of suitable DNA polymerases include the DNA polymerases derived from organisms including thermophilic bacteria, archaebacteria, retroviruses, yeasts, Neurosporas, Drosophilas, primates and rodents.

Studies performed with the total cell RNA follow the differential display protocol of Liang and Pardee (Science, 257:967-970 (1992)). In some embodiments, they are modified by using 5′ biotinylated primers for nonisotopic reverse transcriptase product detection. In these studies, 0.2 μg of total cell RNA are primed for reverse transcription with an anchored primer/probe according to the present invention. Reverse transcription is performed with 200 units of MMLV (Moloney Murine Leukemia Virus) reverse transcriptase (GIBCO/BRL) in the presence of 50 mM Tris-HCl (pH 8.3), 75 mM KCl, 3 mM MgCl₂, 10 mM DTT, 500 μM dNTP, 1 μM anchored primer and 1 U/μl RNase inhibitor. The reaction mixture is incubated at room temperature for 10 minutes, then at 37° C. for 50 minutes. After reverse transcription the enzyme is denatured by heating to 65° C. for 10 minutes.

Reverse transcriptase is an RNA-dependent DNA polymerase requiring a DNA primer and an RNA template to synthesize a complementary DNA strand. For long cDNA chain synthesis, the fast processive activity of the standard reverse transcriptase (RT) enzymes is necessary to produce the lengths required. Processive RT enzymes are those enzymes that perform reverse transcription of RNA to the first strand of cDNA. Traditionally, such RT enzymes include MMLV RT, AMV RT and various others. More recently, certain processive enzymes have been developed that are superior for the production of full-length cDNAs, even at the lower temperature ranges. For example, the MMLV gene has been mutated in order to eliminate the endogenous RNase H activity and this modified enzyme referred to as Superscript RT (Gibco-BRL) is superior for the production of full-length cDNAs. Superscript™ II RNase H⁻ Reverse Transcriptase (U.S. Pat. No. 5,244,797, incorporated herein by reference) is purified to near homogeneity from E. coli containing the pol gene of Moloney Murine Leukemia Virus. The enzyme is used to synthesize first strand cDNA and will generally give higher yields of cDNA and more full length product than other reverse transcriptases. MoMLV RT has a weaker intrinsic RNase H activity than Avian Mycloblastosis Virus (AMV) reverse transcriptase. AMV Reverse Transcriptase has activity at high temperature (42-50° C.). While a reverse transcriptase lacking RNase H activity may be preferred for some embodiments, the methods of this invention do not require RNase H⁻ reverse transcriptase.

### EXAMPLE 4

Reverse Transcription using Thermostable RT

In one embodiment of the invention, thermostable reverse transcriptase (RT) enzymes are used for linear amplification of the detectable population through a series of denaturation and renaturation steps. Further, use of a thermally stable polymerase allows reverse transcription to take place at an elevated temperature, minimizing the effects of RNA secondary structure and thus allowing the incorporation of additional detectable labels.

Thermostable RT enzymes have been developed from various sources. See U.S. Pat. No. 6,406,891 (Legerski). Thermostable enzymes are those enzymes that perform reverse transcription of RNA to the first strand of cDNA at temperatures higher than those used by standard RT enzymes. There are a number of RT enzymes that have temperature optima that range from between about 55° C. to about 90° C. For example, RetroAmp™ is operative at temperatures of 70° C. and above.

Retrotherm™ RT (Epicentre technologies) has both RNA- and DNA-dependent DNA polymerase activities under the same reaction conditions and has no RNase H activity. RetroAmp™ RT DNA Polymerase (Epicentre Technologies), is a highly efficient, thermally stable enzyme. Thermoscript™ (Gibco-BRL) is an avian reverse transcriptase that has been shown to be useful for high temperature cDNA synthesis to improve RT-PCR. GeneAmp Thermostable rTth Reverse Transcriptase (Perkin-Elmer) catalyses the reverse transcription of RNA to cDNA at elevated temperature (60-70° C.) and subsequently amplifies cDNA using the same recombinant thermostable enzyme—rTth DNA polymerase.

The use of a thermal stable polymerase allows reverse transcription to take place at an elevated temperature, minimizing the effects of RNA secondary structure. RetroAmp™ is available in a commercial preparation with a 10×PCR Enhancer (with betaine) referred to as MasterAmp™. The presence of betaine (trimethyl glycine) in the MasterAmp 10×PCR Enhancer substantially improves the yield and specificity of amplification of many target sequences, especially those containing a high G+C content or secondary structure. Betaine lowers the melting temperature of G+C rich regions to a temperature more similar to A+T(U) rich regions. This results in destabilization of double-stranded regions which limits polymerase pausing, thereby increasing the yield of full-length product. In addition, betaine also may enhance the process by protecting DNA polymerases from thermal denaturation.

Typically in the reverse transcriptase reaction, 50 μl reactions are assembled on ice as two separate 25 μl premixes and combined just before the reverse transcription step to minimize RNA sample degradation. One premix includes the dNTPs, primers, and the RNA template. The other premix included all other reaction components. The reactions contain 1×RT-PCR Buffer that comprises 3.0 mM MgCl₂, 1× MasterAmp PCR Enhancer, 0.5 mM MnSO₄, 400 μM each dNTP, 12.5 pmols of each primer, 100 ng of total RNA template, and 2.5 units of RetroAmp™ RT DNA Polymerase. Standard reactions are incubated at 60° C. for 20 minutes for first strand cDNA synthesis. Annealing temperatures vary depending on the primer pair used: typically samples are denatured at 92° C. for 30 seconds, annealed at 60° C. for 30-60 seconds, and extended at 72° C. for 60 seconds. In some embodiments, the reverse transcribed DNA may be subjected to PCR amplification for 20-35 cycles. For example, following RT inactivation, RNA is degraded by an RNase H. Then, PCR is performed where 20 μl cDNA reaction mixture is added to a 50 μl PCR mixture and incubated for 2 minutes at 94° C. PCR conditions involved 35 cycles of 94° C. for 30 s, 55-60° for 30 s, and 68-72° for 1 to 15 minutes. Polymerases used for this method were Platinum Taq™ and eLONGase®.

The ability of GeneAmp Thermostable rTth Reverse Transcriptase (Perkin-Elmer) to efficiently reverse transcribe RNA templates at 70° C. is useful in the present invention because the secondary structures are unstable at the higher reaction temperatures. An additional advantage of performing reverse transcription at higher temperatures is increased specificity of primer hybridization and subsequent extension by the rTth DNA polymerase and therefore sensitivity of the reaction.

### EXAMPLE 5

Labeling During Reverse Transcription on Microchip

Hybridization was carried out on a HO5™ microarray chip (Mergen Ltd., San Leandro, Calif.) which contains approximately 12,000 features. Each feature is a homogeneous population of oligonucleotides, each 30 nucleotides in length and designed to hybridize to a sequence complementary to a specific RNA strand. These 30-mers were synthesized off the chip and a hydrocarbon linker with a terminal amine group was attached to the 5′ end of the oligonucleotides. The oligonucleotides are thus covalently attached to an aldehyde residue on the substrate by a Schiff base formed via the 5′ end of the oligonucleotide, leaving the 3′ end available for primer extension by reverse transcriptase. In a preferred embodiment, the oligonucleotides comprise a sequence of the complementary “antisense” strand such that the oligonucleotides could hybridize directly to the naturally-occurring “sense” RNA strand.

Sense and antisense copy RNA (cRNA) transcripts were synthesized from linearized cDNA templates using Riboprobe Combination System T3/T7 (Promega), according to manufacturer's conditions. The oligonucleotides on the microarray were hybridized with 1 μg cRNA from a human ovarian carcinoma (OVC) cell line in 550 μl of a standard buffer solution which provides a suitable environment for stable yet specific duplexes to form at the hybridization temperature of 30° C. Hybridization was performed overnight at 30° C. A cRNA comprises a sequence complementary to a naturally occurring RNA (e.g., mRNA) and was made using a standard procedure familiar to one knowledgeable in the art of molecular biology. (See Sambrook et al., supra). In a preferred embodiment, where the microarray is composed of oligonucleotides complementary to the sense strand, mRNA can be used directly for hybridization to the microarray without prior conversion to cRNA.

Following hybridization, the microarray was washed with constant shaking at 60 rpm, in preheated washing solutions according to the following schedule:

| | | |
|---|---|---|
| 2.0X SSC + 0.1% SDS | at 42° C. | for 10 min twice |
| 0.2X SSC | at 37° C. | for 10 min |
| 0.1X SSC | at 37° C. | for 10 min |

Reverse transcription (RT) reaction was performed on the array in a reaction mixture comprising:

| | |
|---|---|
| 4 μl | 5x RT buffer |
| 2 μl | 0.1 M dithiothreitol (DTT) |
| 2 μl | AMV reverse transcriptase |
| 1 μl | 10 mM dATP |
| 1 μl | 10 mM dGTP |
| 1 μl | 10 mM dTTP |
| 6 μl | 1 mM dCTP |
| 3 μl | 1 mM biotin-14-dCTP |

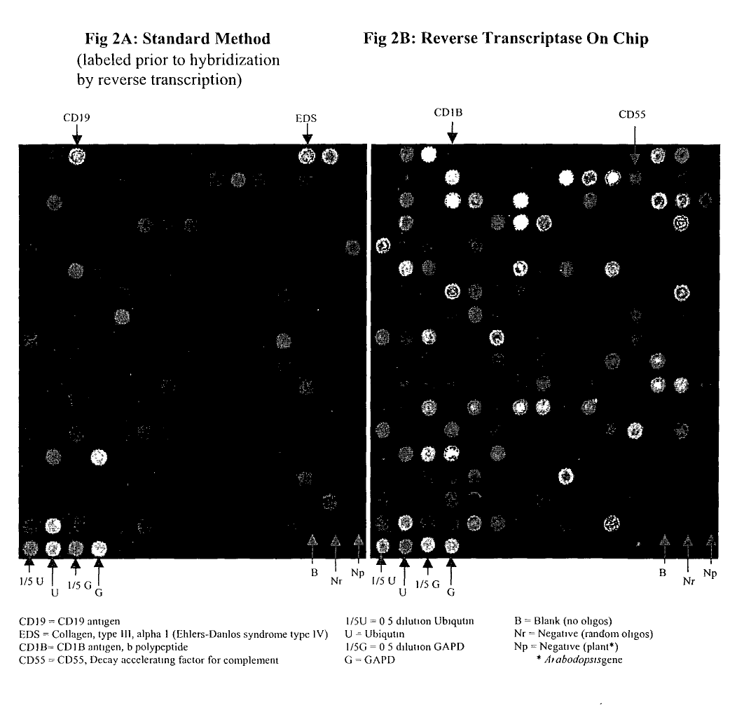

In different embodiments the amount of biotin-14-dCTP can be varied according to the desired sensitivity. The reaction mixture was applied to the array, which was then covered with a coverslip and incubated at 42° C. for one hour in a humidity chamber. The array was blocked for 30 minutes at room temperature with constant rotation on an orbital shaker at 50 rpm in a milk-based blocking buffer. A streptavidin solution was applied to the array and incubated at 4° C. for 30 minutes with constant rotation on an orbital shaker. The array was then washed in a solution of malic acid and Tween-20™ (standard washing buffer) three times for 15 minutes each at room temperature. The array was incubated with anti-streptavidin antibody-Cy3 solution while gently rotating on an orbital shaker at 4° C. for 30 min. Washes with malic acid and Tween-20™ buffer were repeated several times. The array was then dried and scanned for Cy3. A visualization of the gene-expression profile is shown in FIG. 2B.

For comparison, 20 μg biotinylated human ovarian carcinoma (OVC) cell line RNA was isolated by standard procedures and a first cDNA strand was formed using a polyT-T7 primer and reverse transcriptase. The cDNA strand was then used as a template to create double-stranded cDNA employing a commercially available kit from Roche Diagnostics. Biotin-labeled probe was made by in vitro transcription using T7 in the presence of biotin-14-dCTP. The biotinylated RNA was hybridized to an identical array and the gene expression profile visualized (FIG., 2A) by standard procedures.

A representative sub-grid (one of 32) is shown in FIGS. 2A-2B illustrating the results for both methods. Identified by arrows on the grids are signals resulting from test oligonucleotides and negative control oligonucleotides used according to Table 1. As can be seen from FIGS. 2A and 2B, the results are comparable and any apparent differences may be due to differences in sensitivity.

TABLE 1
Samples identified on microarrays in FIGS. 2A and 2B.

| Symbol | Test Oligonucleotides |
|---|---|
| CD19 | CD19 antigen |
| EDS | Collagen, type III, alpha 1 (Ehlers-Danlos syndrome type IV) |
| CD1B | CD1B antigen, b polypeptide |
| CD55 | CD55, Decay accelerating factor for complement |
| Symbol | Control Oligonucleotides |
| ⅕U | 0.2x dilution of Ubiqutin |
| U | Ubiqutin |
| ⅕G | 0.2x dilution of GAPD |
| G | GAPD |
| B | Blank (no oligonucleotides) |
| Nr | Negative (random oligonucleotides) |
| Np | Negative (plant* gene) * Arabidopsis |

### EXAMPLE 6

Labeling for Multiple Cycles using Thermostable Reverse Transcription

Hybridization is carried out on a HO5™ microarray chip (Mergen Ltd., San Leandro, Calif.) which contains approximately 12,000 features. Each feature is a homogeneous population of oligonucleotides, each 30 nucleotides in length and designed to hybridize to a sequence complementary to a specific RNA strand. These 30-mers are synthesized off the chip and a hydrocarbon linker with a terminal amine group is attached to the 5′ end of the oligonucleotides. The oligonucleotides are thus covalently attached to an aldehyde residue on the substrate by a Schiff base formed via the 5′ end of the oligonucleotide, leaving the 3′ end available for primer extension by reverse transcriptase. In a preferred embodiment, the oligonucleotides comprise a sequence of the complementary “antisense” strand such that the oligonucleotides could hybridize directly to the naturally-occurring “sense” RNA strand.

Sense and antisense copy RNA (cRNA) transcripts are synthesized from linearized cDNA templates using Riboprobe Combination System T3/T7 (Promega), according to manufacturer's specified conditions. The oligonucleotides on the microarray are hybridized with 1 μg cRNA from a human ovarian carcinoma (OVC) cell line in 550 μl of a standard buffer solution which provides a suitable environment for stable yet specific duplexes to form at the hybridization temperature of 30° C. Hybridization is performed overnight at 30° C. In a preferred embodiment, where the microarray is composed of oligonucleotides complementary to the sense strand, mRNA from cells can be used directly for hybridization to the microarray without prior conversion to cRNA.

Following hybridization, the microarray is washed with constant shaking at 60 rpm, in preheated washing solutions according to the following schedule:

| | | |
|---|---|---|
| 2.0X SSC + 0.1% SDS | at 42° C. | for 10 min twice |
| 0.2X SSC | at 37° C. | for 10 min |
| 0.1X SSC | at 37° C. | for 10 min |

Reverse transcription (RT) reaction is then performed on the array in a reaction mixture comprising:

| | |
|---|---|
| 4 μl | 5x RT buffer |
| 2 μl | 0.1 M dithiothreitol (DTT) |
| 2 μl | RetroAmp ™ thermostable RT(Epicentre; or AMV RT) |
| 1 μl | 10 mM dATP |
| 1 μl | 10 mM dGTP |
| 1 μl | 10 mM dTTP |
| 6 μl | 1 mM dCTP |
| 3 μl | 1 mM biotin-14-dCTP |

In different embodiments the amount of biotin-14-dCTP can be varied according to the desired sensitivity. The reaction mixture is applied to the array, which is then covered with a coverslip and incubated at 42° C. for one hour in a humidity chamber.

Two or more cycles of the following protocol are performed to increase sensitivity of the assay:

| | | | |
|---|---|---|---|
| (a) | maintain at | 94° C. | for 2 min.; |
| (b) | maintain at | 4° C. | for 5 min.; and |
| (c) | maintain at | 42° C. | for 60 min. |

Where AMV reverse transcriptase is used in the initial reaction, the enzyme (AMV RT) can be replenished between steps (b) and (c). Preferably, a thermostable RT is used for this assay. In some embodiments, a ribonuclease inhibitor (such as RNasin™) is added to the reaction mixture or an RNase-deficient thermostable reverse transcriptase is used to increase stability of the RNA template during the process. The array is blocked for 30 minutes at room temperature with constant rotation on an orbital shaker at 50 rpm in a milk-based blocking buffer. A streptavidin solution is applied to the array and incubated at 4° C. for 30 minutes with constant rotation on an orbital shaker. The array is then washed in a solution of malic acid and Tween-20™ (standard washing buffer) three times for 15 minutes each at room temperature. The array is incubated with anti-streptavidin antibody-Cy3 solution while gently rotating on an orbital shaker at 4° C. for 30 min. Washes with malic acid and Tween-20™ buffer are repeated several times. The array is then dried and scanned for Cy3.

In some embodiments, GeneAmp Thermostable rTth Reverse Transcriptase (Perkin-Elmer) is used to catalyze the reverse transcription of RNA to cDNA at elevated temperature (60-70° C.) and subsequent to the addition of appropriate primers (such as oligo dT), the cDNA strand is amplified using the same thermostable enzyme—rTth DNA polymerase.

All publications, patents and patent applications mentioned in this specification are hereby incorporated by reference into the specification in their entirety for all purposes.

Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

The foregoing is offered primarily for purposes of illustration. It will be readily apparent to those of ordinary skill in the art that the operating conditions, materials, procedural steps and other parameters of the invention described herein may be further modified or substituted in various ways without departing from the spirit and scope of the invention. Thus, the preceding description of the invention should not be viewed as limiting but as merely exemplary.

We claim:

1. A method for detecting a target RNA in a sample, the method comprising,
(a) providing an array comprising a DNA primer comprising a sequence complementary to the target RNA;
(b) contacting the primer with an RNA sample under conditions that promote specific hybridization between the primer and the target RNA;
(c) incubating the primer-RNA heteroduplex with a reverse transcriptase enzyme, under conditions that allow reverse transcription of the RNA into cDNA such that a detectable label is incorporated into the cDNA; and
(d) detecting the detectable label incorporated into the nascent cDNA strand synthesized by the reverse transcriptase, wherein the presence of the label in the cDNA is indicative of the presence of the target RNA in the sample.

2. The method of claim 1, further comprising:
(e) comparing the presence or absence of a target RNA in a sample cell with respect to that in a reference cell.

3. The method of claim 1, further comprising:
(e) determining a level of target RNA in a sample cell and comparing with the level in a reference cell.

4. The method of claim 1, wherein a plurality of DNA primers is provided for detecting a plurality of target RNAs in a sample, wherein each primer is complementary to a different target RNA molecule and further wherein each primer is located at a distinct and identifiable location on a solid support.

5. The method of claim 1, wherein the detectable label is selected from the group consisting of a radiolabeled molecule, a fluorescent molecule, and a chromogenic molecule.

6. The method of claim 5, wherein the detectable label is incorporated into a modified nucleotide, further wherein the modified nucleotide is not a chain-terminating nucleotide.

7. The method of claim 6, wherein the detectable label is at least one of radiolabeled-dNTP, fluoro-dNTP, biotinylated dNTP or a digoxigenin-dNTP.

8. The method of claim 1, wherein the detectable label is conjugated to a molecule that binds a second label incorporated into the nascent polynucleotide.

9. The method of claim 1, wherein the array comprises from at least about 100 to at least about 1,000,000 primers.

10. The method of claim 9, wherein the array comprises at least about 1,000 primers.

11. The method of claim 1, wherein the sample is derived from a cell and the presence of at least one target RNA is indicative of a condition selected from the group consisting of an infection, a disease state, a predisposition to a disease state, a developmental, a physical, a chemical and a biological state.

12. The method of claim 1, wherein the sample is derived from a cell and the absence of at least one target RNA is indicative of a condition selected from the group consisting of an infection, a disease state, a predisposition to a disease state, a developmental, a physical, a chemical and a biological state.

13. The method of claim 1, wherein the primer is immobilized on a substrate.

14. The method of claim 13, wherein the primer is immobilized on the substrate by a covalent bond, such that the 3′ end of the primer is free.

15. The method of claim 14, wherein the covalent bond is selected from the group consisting of a Schiff base, a photocleavable bond, an electrostatic bond, a disulfide bond, a peptide bond, a diester bond, and a selectively releasable bond.

16. The method of claim 13, wherein the primer is immobilized on the substrate by a non-covalent coupling, such that the 3′ end of the primer is free.

17. The method of claim 16, wherein the non-covalent coupling is selected from the group consisting of an electrostatic interaction, a hydrogen bonds, an antibody-antigen coupling, a biotin-avidin interaction, a biotin-streptavidin interaction, a Staphylococcus aureus protein A-IgG antibody F_{c} fragment interaction, and a streptavidin/protein A chimera.

18. The method of claim 13, wherein the substrate is selected from the group consisting of a plastic, a ceramic, a nylon, a polyester, a metal, a resin, a gel, a membrane, a nitrocellulose membrane, a plate, a bead, a thin film, a glass, a cylinder, a tagged bead, a magnetic bead, an optical fiber, and a woven fiber.

19. The method of claim 1, wherein the RNA sample is isolated from a biological source.

20. The method of claim 19, wherein the RNA sample is amplified following isolation from a biological source.

21. The method of claim 1, wherein the RNA sample is prepared by transcription in vitro.

22. The method of claim 1, wherein the RNA sample comprises at least one expression control RNA.

23. The method of claim 1, wherein the primer further comprises a sequence for a promoter of a DNA-dependent RNA polymerase.

24. The method of claim 23, wherein the DNA-dependent RNA polymerase is selected from the group consisting of T3 RNA polymerase, T7 RNA polymerase, and SP6 RNA polymerase.

25. The method of claim 1, further comprising:
(e) amplifying the cDNA strand by a polymerase chain reaction (PCR) performed in situ.

26. The method of claim 25, wherein the PCR reaction is performed using a Taq DNA polymerase, a Tth1 DNA polymerase, a Vent DNA polymerase, a Pfu DNA polymerase, or a thermostable reverse transcriptase.

27. A method for detecting a target RNA in a sample, the method comprising,
(a) providing a microarray comprising at least one DNA primer comprising a sequence complementary to a sequence of the target RNA;
(b) contacting the microarray with an RNA sample under conditions that allow specific hybridization between the primer and the target RNA;
(c) incubating the primer-RNA heteroduplex with a reverse transcriptase enzyme, under conditions that allow reverse transcription of the RNA into cDNA; and
(d) detecting the cDNA-RNA heteroduplex synthesized by the reverse transcriptase enzyme, wherein presence of the cDNA-RNA heteroduplex is indicative of the presence of the target RNA in the sample, wherein the detection is not mediated by an antibody against a DNA-RNA hybrid.

28. The method of claim 27, wherein the detectable label is conjugated to a molecule that binds the nascent polynucleotide.

29. The method of claim 27, further comprising:
(e) providing a thermostable reverse transcriptase; and
(f) performing a plurality of cycles of primer extension reactions under conditions wherein the temperature exceeds the melting temperature of a cDNA-RNA hybrid between each cycle.

30. The method of claim 27, wherein the cDNA-RNA heteroduplex is detected by a detectable reagent that specifically binds a double stranded polynucleotide.

31. The method of claim 30, wherein the detectable reagent is selected from the group consisting of an intercalating compound, a polynucleotide duplex-dependent fluorescence quenching compound, DAPI, ethidium bromide, thiazole orange, bis-benzimide and acridine orange.

32. The method of claim 30, wherein the detectable reagent is indirectly detectable by a second detectable molecule which binds the reagent.

33. The method of claim 27, wherein a plurality of DNA primers is provided for detecting a plurality of target RNAs in a sample, wherein each primer is complementary to a different target RNA molecule and further wherein each primer is located at a distinct and identifiable location on a solid support.

34. The method of claim 27, wherein the array comprises from at least about 100 to at least about 1,000,000 primers.

35. The method of claim 34, wherein the array comprises at least about 1,000 primers.

36. The method of claim 27, wherein the sample is derived from a cell and the presence of at least one target RNA is indicative of a condition selected from the group consisting of an infection, a disease state, a predisposition to a disease state, a developmental, a physical, a chemical and a biological state.

37. The method of claim 27, wherein the sample is derived from a cell and the absence of at least one target RNA is indicative of a condition selected from the group consisting of an infection, a disease state, a predisposition to a disease state, a developmental, a physical, a chemical and a biological state.

38. The method of claim 27, wherein the primer is immobilized on the substrate by a covalent bond, such that the 3′ end of the primer is free.

39. The method of claim 38, wherein the covalent bond is selected from the group consisting of a photocleavable bond, an electrostatic bond, a disulfide bond, a peptide bond, a diester bond, and a selectively releasable bond.

40. The method of claim 27, wherein the primer is immobilized on the substrate by a non-covalent coupling, such that the 3′ end of the primer is free.

41. The method of claim 40, wherein the non-covalent coupling is selected from the group consisting of an electrostatic interaction, a hydrogen bonds, an antibody-antigen coupling, a biotin-avidin interaction, a biotin-streptavidin interaction, a Staphylococcus aureus protein A-IgG antibody F_{c} fragment interaction, and a streptavidin/protein A chimera.

42. The method of claim 13, wherein the substrate is selected from the group consisting of a plastic, a ceramic, a nylon, a polyester, a metal, a resin, a gel, a membrane, a nitrocellulose membrane, a plate, a bead, a thin film, a glass, a cylinder, a tagged bead, a magnetic bead, an optical fiber, and a woven fiber.

43. The method of claim 27, wherein the RNA sample is isolated from a biological source.

44. The method of claim 43, wherein the RNA sample is amplified following isolation from a biological source.

45. The method of claim 27, wherein the RNA sample is prepared by transcription in vitro.

46. The method of claim 27, wherein the RNA sample comprises at least one expression control RNA.

47. A method for detecting a target RNA in a sample, the method comprising,
(a) providing an array comprising a DNA primer comprising a sequence complementary to the target RNA;
(b) contacting the primer with an RNA sample under conditions that promote specific hybridization between the primer and the target RNA;
(c) incubating the primer-RNA heteroduplex with a thermostable reverse transcriptase enzyme, under conditions that allow reverse transcription of the RNA into cDNA such that a detectable label is incorporated into the cDNA;
(d) performing at least two cycles of hybridization and reverse transcription, under conditions comprising a temperature higher than a melting temperature of the RNA:cDNA duplex; and
(e) detecting the detectable label incorporated into the nascent cDNA strand synthesized by the reverse transcriptase, wherein the presence of the label in the cDNA is indicative of the presence of the target RNA in the sample.

48. The method of claim 47, further comprising:
(f) comparing the presence or absence of a target RNA in a sample cell with respect to that in a reference cell.

49. The method of claim 47, wherein the hybridization and reverse transcription steps are performed in the presence of an RNase H inhibitor.

50. The method of claim 47, wherein the thermostable reverse transcriptase lacks an RNase H function.

51. A kit for identifying sequence variations in a target polynucleotide as compared to a reference sequence, comprising:
a) an array of at least two oligonucleotide primers immobilized on a solid phase support, wherein each oligonucleotide primer is selected to comprise a sequence complementary to a target RNA, and occupies an identifiable and discrete area of the array;
b) reagents suitable for a reverse transcription reaction on the array; and
c) detection means for detecting a nascent cDNA strand on the array.

52. The kit of claim 51, wherein the detection means comprises a detectable label that is incorporated into the nascent cDNA strand during the reverse transcription reaction.

53. The kit of claim 52, wherein the detectable label is a fluorescent molecule.

54. The kit of claim 52, wherein the detectable label comprises at least one of radiolabeled-dNTP, fluoro-dNTP, biotinylated dNTP or a digoxigenin-dNTP.
